(19) United States
(12) Patent Application Publication
(10) Pub. No.: US 2026/0348946 A1
(43) Pub. Date: Nov. 5, 2026

(54) A HUMAN MODEL FOR HEART FAILURE WITH PRESERVED EJECTION FRACTION (HFPEF) FOR THE DISCOVERY OF THERAPEUTICS AND THERAPEUTIC TARGETS

(71) Applicant: NOVOHEART INTERNATIONAL LIMITED, Hong Kong (CN)

(72) Inventors: Ronald A. Li, Hong Kong (CN); Roger J. Hajjar, Hong Kong (CN)

(21) Appl. No.: 19/099,982

(22) PCT Filed: Aug. 4, 2023
(86) PCT No.: PCT/IB2023/000464

§ 371 Date: Jan. 30, 2025

Related U.S. Application Data
(60) Provisional application No. 63/395,225, filed on Aug. 4, 2022.

(51) Int. Cl. G01N 33/50

(52) U.S. Cl. CPC G01N 33/5082; G01N 2333/495; G01N 2333/5754

(57) ABSTRACT

The disclosure provides methods for identifying a therapeutic for treatment of heart failure with preserved ejection fraction. The method may comprise contacting cardiac tissue or a cardiac organoid with an effective amount of Transforming Growth Factor-pi and an effective amount of Endothelin-1 (ET-1) to induce heart failure with preserved ejection fraction in the cardiac tissue, exposing the cardiac tissue or cardiac organoid exhibiting heart failure with preserved ejection fraction to a candidate therapeutic, and identifying the candidate therapeutic as a therapeutic for heart failure with preserved ejection fraction if the cardiac tissue or cardiac organoid if the candidate therapeutic induces a reduction in any one or more of the following characteristics of the cardiac tissue or cardiac organoid: passive strain, stiffness, cardiomyocyte hypertrophy, or fibrosis. In some embodiments, the candidate therapeutic identified as a therapeutic for treatment of cardiac failure with preserved ejection fraction induces an increase in contractile kinetics or relaxation kinetics. Additional embodiments of the disclosed methods identify therapeutics that induce any combination of the aforementioned altered characteristics of cardiac tissue and/or cardiac organoids.

### FIELD

The disclosure relates generally to the fields of medicine and healthcare, and more particularly to the field of cardiovascular healthcare.

### BACKGROUND

Heart failure with preserved ejection fraction (HFpEF) is becoming the most prevalent cause of heart failure [1-3]. It makes up more than 50% of all heart failure cases. In contrast to numerous other cardiovascular diseases, its prevalence is increasing, and the increasing incidence is resulting in substantial increases in morbidity, mortality, and costs to society [1-4]. HFpEF affects the elderly in an age-dependent fashion, and with the aging of the population, its prevalence is increasing in Europe and the US. Mortality from HFpEF is high and is comparable to heart failure with reduced ejection fraction (HFrEF).

Unfortunately, many of the therapies that reduce mortality and morbidity in HFrEF have little effect on HFpEF. HFpEF patients do not respond to standard-of-care for HFrEF because the biology and the clinical course for the two diseases are very different. The main clinical presentation of patients with HFpEF is exercise intolerance and dyspnea on exertion. Such dyspnea can be profound, can occur at very low levels of exertion, and can thereby limit physical activity, leading to a downward spiral of inactivity and deconditioning that compounds the underlying cardiac disease. Physiologically, HFpEF is characterized by 1) delayed and incomplete left ventricle (LV) relaxation, 2) reduced LV compliance, 3) atrial-ventricular mismatch (AV coupling mismatch), 4) abnormal ventriculo-aortic coupling, and 5) chronotropic incompetence. The exact mechanisms underlying this fundamental morbid symptom, however, are unclear.

Several animal experimental models including mouse, rats and pigs have been established and reported to mimic the disease Heart Failure with preserved Ejection Fraction (HFpEF),

There are several problems/deficiencies with the way that the existing technology works. Existing models of HFpEF are not of human origin and therefore fail to recapitulate all the key characteristic clinical manifestations due to species-specific and other inherent differences (e.g., mouse hearts are significantly smaller in size with 10-fold higher beating rates, pigs have similar cardiac anatomy to humans but substantially different responses to pharmacological responses).

Heart failure with preserved ejection fraction (HFpEF) makes up more than 50% of all heart failure cases. In contrast to numerous other cardiovascular diseases, its prevalence is increasing, resulting in substantial morbidity, mortality, and cost to society. HFpEF affects the elderly in an age-dependent fashion, and with the aging of the population, its prevalence is increasing in Europe and the US. Mortality from HFpEF is high and is comparable to heart failure with reduced ejection fraction (HFrEF). Unfortunately, many of the therapies that reduce mortality and morbidity in HFrEF have little effect in HFpEF. HFpEF patients do not respond to standard-of-care procedures for HFrEF because the biology and the clinical course for the two diseases are very different.

Two pathological characteristics have been shown to be markedly prevalent in patients with HFpEF: cardiac myocyte hypertrophy and cardiac fibrosis. In a large, prospective myocardial tissue analysis of HFpEF, myocardial fibrosis and hypertrophy were the common features [11, 12]. During the last decade, significant effort has focused on the elucidation of the signaling pathways that mediate the complex response of cardiomyocytes to various hypertrophic stimuli and the progression from cardiac hypertrophy to heart failure. Endothelin-1 has been shown to be a potent hypertrophic stimulator of cardiac myocytes and has been shown to be elevated in patients with HFpEF [22]. Cardiac fibrosis (CF) is highly associated with heart failure (HF) and especially with HFpEF. Although CF was traditionally regarded as a secondary phenomenon, it was recently proposed to play a primary role in the progression of HFpEF [23]. The clinical outcome of patients with severe aortic stenosis undergoing aortic valve replacements correlated with CF severity [24]. In line with this finding, CF, was associated with mortality in patients with HFpEF [25]. Activation of neurohumoral pathways stimulates fibroblasts directly. Members of the Transforming Growth Factor-β family, which are secreted in the cardiac interstitium, play a specific role in activating specific aspects of the fibrotic response. Secreted fibrogenic mediators and matricellular proteins bind to cell surface receptors in fibroblasts and induce the synthesis, processing and metabolism of the extracellular matrix.

There is an urgent need to better understand the underlying cellular and molecular processes of HFpEF, so as to develop targeted therapies for this largely untreated patient population. Although several animal experimental models have been established and reported to mimic the human disease, they are not of human origin and therefore fail to recapitulate all the key characteristic clinical manifestations due to species-specific and other inherent differences (e.g., mouse hearts are significantly smaller in size, with 10-fold higher beating rates, pigs have similar cardiac anatomy to humans but substantially different responses to pharmacological agents).

Thus, a need continues to exist in the art for methods of treating heart failure with preserved ejection fraction and, consequently, a need continues to exist in the art for therapeutics and therapeutic targets to treat the condition as well as robust and accurate models for use in screening methods for identifying such therapeutics and targets.

### SUMMARY

We report the construction of the first ex vivo human HFpEF heart models by using a combination of state-of-the-art human pluripotent stem cells (hPSCs) in a cardiac tissue engineering approach, with the hPSCs directed to differentiate into human ventricular cardiomyocytes (hvCMs) for assembling into engineered tissues: The approach involves human ventricular cardiac tissue strips (hvCTS) and a three-dimensional (3D) electro-mechanically coupled, fluid-ejecting miniature human ventricle-like cardiac organoid chamber (hvCOC) that faithfully reproduces the pathological characteristics that have been shown to be markedly prevalent in patients with HFpEF, i.e., cardiac myocyte hypertrophy and cardiac fibrosis. The disclosed materials, methods and systems are useful as a tool for drug discovery and for pre-clinical in vitro testing of drug and therapy candidates.

Three-dimensional bioengineered tissue constructs have been developed using cardiomyocytes derived from human pluripotent stem cells. They allow modeling of different cardiovascular disease states and testing of drugs and biologics in human tissues. The ex vivo human HFpEF heart models disclosed herein have been extensively validated using a range of phenotypic assessments. Human PSCs (HES2: human embryonic stem cell; ESI, NIH code ES02 and L-EdV: human induced pluripotent stem cells) were directed to differentiate into human ventricular cardiomyocytes (hvCMs) for assembling into engineered tissues in the form of human ventricular cardiac tissue strips (hvCTS) and a three-dimensional (3D) electro-mechanically coupled, fluid-ejecting miniature human ventricle-like cardiac organoid chamber (hvCOC) [6-9].

The disclosure provides materials and methods that exhibit new, unique features. In contrast to existing technologies, the disclosure provides for the use of biologics for induction of disease without need for patient specific human induced pluripotent stem cells In addition, the disclosed model is amenable to sophisticated phenotypic measurements and capable of recapitulating characteristic phenotypes seen in patients. In some exemplary embodiments, the HFpEF phenotype is rescued (i.e., the HFpEF condition is reversed) with an adeno-associated virus gene therapy AAV1-SERCA in engineered tissues, but the disclosed model is suitable for assessing the abilities of any agent or candidate agent to rescue or improve a HFpEF condition.

One aspect of the disclosure is directed to a method for identifying a therapeutic for treatment of heart failure with preserved ejection fraction, the method comprising: (a) contacting cardiac tissue with an effective amount of Transforming Growth Factor-31 and an effective amount of Endothelin-1 (ET-1) to induce heart failure with preserved ejection fraction in the cardiac tissue; (b) exposing the cardiac tissue exhibiting heart failure with preserved ejection fraction to a candidate therapeutic, and (c) identifying the candidate therapeutic as a therapeutic for heart failure with preserved ejection fraction if the level of (i) passive strain of the cardiac tissue, (ii) cardiac tissue stiffness, (iii) cardiac myocyte hypertrophy, or (iv) cardiac fibrosis, is reduced, or the (v) contractile kinetics, or (vi) relaxation kinetics are increased, or any combination thereof, in the presence of the candidate therapeutic compared to the absence of the candidate therapeutic. In some embodiments, the cardiac tissue is formed into a cardiac tissue strip. In some embodiments, the cardiac tissue strip is formed between two posts and the reduced level of passive strain is detected by a reduction in the bending of the cardiac tissue strip. In some embodiments, the candidate therapeutic reduces the passive strain of the cardiac tissue. In some embodiments, the candidate therapeutic reduces the tissue stiffness of the cardiac tissue. In some embodiments, the candidate therapeutic reduces cardiac myocyte hypertrophy in the cardiac tissue. In some embodiments, the candidate therapeutic reduces cardiac fibrosis in the cardiac tissue. In some embodiments, the candidate therapeutic increases the contractile kinetics of the cardiac tissue. In some embodiments, the candidate therapeutic increases the relaxation kinetics of the cardiac tissue. In some embodiments, the candidate therapeutic is a small molecule, a nucleic acid, or a cell.

A related aspect of the disclosure is drawn to a method for identifying a therapeutic for treatment of heart failure with preserved ejection fraction, the method comprising: (a) contacting a cardiac organoid with an effective amount of Transforming Growth Factor-β1 and an effective amount of Endothelin-1 (ET-1) to induce heart failure with preserved ejection fraction in the cardiac tissue; (b) exposing the cardiac organoid exhibiting heart failure with preserved ejection fraction to a candidate therapeutic, and (c) identifying the candidate therapeutic as a therapeutic for heart failure with preserved ejection fraction if the level of (i) passive strain of the cardiac organoid, (ii) stiffness of cardiac tissue in the cardiac organoid, (iii) cardiac myocyte hypertrophy in the cardiac organoid, or (iv) fibrosis in the cardiac organoid, is reduced, or the (v) contractile kinetics of the cardiac organoid, or (vi) relaxation kinetics of the cardiac organoid are increased, or any combination thereof, in the presence of the candidate therapeutic compared to the absence of the candidate therapeutic. In some embodiments, the candidate therapeutic reduces the passive strain of the cardiac organoid. In some embodiments, the candidate therapeutic reduces the stiffness of the cardiac organoid. In some embodiments, the candidate therapeutic reduces cardiac myocyte hypertrophy in the cardiac organoid. In some embodiments, the candidate therapeutic reduces cardiac fibrosis in the cardiac organoid. In some embodiments, the candidate therapeutic increases the contractile kinetics of the cardiac organoid. In some embodiments, the candidate therapeutic increases the relaxation kinetics of the cardiac organoid. In some embodiments, the candidate therapeutic is a small molecule, a nucleic acid, or a cell.

Another aspect of the disclosure is a method of assessing the toxicity of a compound to a cardiomyocyte, comprising: (a) contacting cardiac tissue with an effective amount of Transforming Growth Factor-31 and an effective amount of Endothelin-1 (ET-1) to induce heart failure with preserved ejection fraction in the cardiac tissue; (b) exposing the cardiac tissue exhibiting heart failure with preserved ejection fraction to a compound, and (c) determining or confirming the toxicity of the compound to cardiac tissue if the level of (i) passive strain of the cardiac tissue, (ii) cardiac tissue stiffness, (iii) cardiac myocyte hypertrophy, or (iv) cardiac fibrosis, is reduced, or the (v) contractile kinetics, or (vi) relaxation kinetics are increased, or any combination thereof, in the presence of the compound compared to the absence of the compound. In some embodiments, the toxicity of the compound to the cardiac tissue is determined. In some embodiments, the cardiac tissue is a cardiac tissue strip. In some embodiments, the cardiac tissue is a cardiac organoid.

Yet another aspect of the disclosures is a method of determining the efficacy of a compound in reducing at least one symptom of heart failure with preserved ejection fraction, the method comprising: (a) contacting cardiac tissue with an effective amount of Transforming Growth Factor-β1 and an effective amount of Endothelin-1 (ET-1) to induce heart failure with preserved ejection fraction in the cardiac tissue; (b) exposing the cardiac tissue exhibiting heart failure with preserved ejection fraction to a compound, and (c) determining or confirming the efficacy of the compound in reducing at least one symptom of heart failure with preserved ejection fraction in the cardiac tissue if the level of (i) passive strain of the cardiac tissue, (ii) cardiac tissue stiffness, (iii) cardiac myocyte hypertrophy, or (iv) cardiac fibrosis, is reduced, or the (v) contractile kinetics, or (vi) relaxation kinetics are increased, or any combination thereof, in the presence of the compound compared to the absence of the compound. In some embodiments, the efficacy of the compound in reducing at least one symptom of heart failure with preserved ejection fraction is determined. In some embodiments, the cardiac tissue is a cardiac tissue strip. In some embodiments, the cardiac tissue is a cardiac organoid.

Other features and advantages of the disclosure will become apparent from the following detailed description, including the drawing. It should be understood, however, that the detailed description and the specific examples, while indicating embodiments, are provided for illustration only, because various changes and modifications within the spirit and scope of the disclosure will become apparent to those skilled in the art from the detailed description.

### BRIEF DESCRIPTION OF THE DRAWING

FIG. 1. Functional consequences of the combined effect of ET-1/TGF-β1 on the contractility of hvCTS. (A) Representative images of time-matched hvCTS under control conditions and with combined Endothelin-1/Transforming Growth Factor-31 (ET-1/TGF-β1) treatment. Note that the posts for the treated group were bent, indicating a higher passive strain of the corresponding mounted hvCTS. (B) Representative normalized force tracings recorded from hvCTS of the same groups from (A). The ET-1/TGF-β1-treated group (dashed line) displayed slower contractile and relaxation kinetics than the control group (solid line). (C) Tabulated plots for the systolic force. Control group (solid circles); ET-1/TGF-β1-treated group (dashed line). (D) Contractile time and relaxation times of the same groups from (A)-(B) as recorded by post-tracking measurements. n=4-20. Control group (solid circles); ET-1/TGF-β1-treated group (open circles). ** p<0.01 and *** p<0.001. (E) Stiffness, developed force, max dF/dt and max-dF/dt plotted as a function of % strain for control and ET-1/TGF-β1-treated hvCTS stiffness, recorded using an isometric system for length-control. n=7-12.

FIG. 2. ET-1/TGF-β1 conferred HFpEF phenotypic characteristics on hvCOC. (A) Representative images of time-matched hvCOC under control conditions and with combined ET-1/TGF-β1 treatment. Note the more compact appearance of the treated group, consistent with a stiffer hvCOC. (B) Changes in diastolic pressure were plotted against the changes in diastolic area after hydrostatic loading (100 μl). The slope of the plotted graph represented the stiffness. Control (solid line); HFpEF (dashed line). (C) ET-1/TGF-β1-treated hvCOC (open circles) were significantly stiffer than the control group (solid circles). (D) Representative pressure-volume loops of control (solid line) and ET-1/TGF-β1-treated (dashed line) hvCOCs. (E) Change in pressure (left) and change in volume (right) versus time for control (solid line) and ET-1/TGF-β1-treated (dashed line) hvCOCs. (F) Tabulated plots for the developed pressure, stroke volume, stroke work and ejection fraction for control (solid circles) and ET-1/TGF-β1-treated (open circles) hvCOCs, indicating that the contractile function was not significantly altered despite an increase in stiffness. Collectively, treated hvCOC displayed phenotypes consistent with HFpEF characteristics. (B), (C), and (F) n=8-13. Student t-test. * p<0.05. Higher concentrations do compromise contractility of hvCOC.

FIG. 3. Transcriptomic and bioinformatic analyses of normal and HFpEF patients. The hvCTS and hvCOC heart models revealed that SERCA2a was uniquely downregulated in HFpEF. (A) Venn diagram showing 29% shared differentially expressed genes (DEGs) between HFpEF patients and the HFpEF-hvCTS heart model disclosed herein, and 33% shared DEGs between HFpEF patients and the HFpEF-hvCOC heart model disclosed herein. Comparative functional analyses using Ingenuity Pathway Analysis shows that the cardiac organoids of the HFpEF-hvCOC model were more similar to human HFpEF patients than the cardiac tissue strips of the HFpEF-hvCTS model, based on hierarchical clustering of enriched canonical pathways, biofunctions and cardiotoxicity functions. (B) Calcium Signaling Pathway was similarly enriched in both human HFpEF patients and in both the engineered HFpEF-hvCTS and the HFpEF-hvCOC tissue models and native Human heart tissues from HFpEF patients relative to their corresponding controls. Of note, SERCA2a was the most highly expressed gene in the gene ontology or GO term ‘Regulation of ATPase-coupled calcium transmembrane transport activity’ in normal patients and control engineered hvCTS and models. Interestingly, SERCA2a (ATP2A2) was also most significantly downregulated in HFpEF patients and in both engineered HFpEF-hvCTS and HFpEF-hvCOC heart models compared to their respective controls. These transcriptomic and bioinformatic results were consistent with the HFpEF phenotypes observed in HFpEF patients and in the engineered HFpEF-hvCTS and HFpEF-hvCOC models, indicating that SERCA2A is a target candidate for amelioration, rescue or reversal of the HFpEF disease in humans or symptoms thereof.

FIG. 4. Titer- and time-dependences of AAV transduction. (A) Representative fluorescence and brightfield images of hvCM after 14 days of AAV-GFP transduction with different viral genome (vg) per cardiomyocyte. (B) The effect of time and virus titer on % GFP expressing cells after AAV-GFP transduction. (C) The effect of SERCA gene expression after 14 days of AAV-GFP or AAV1.SERCA2A transduction. (C) n=1−3. One way ANOVA. *p<0.05.

FIG. 5. AAV1-SERCA2a rescue HFpEF phenotype in hvCTS. (A) Representative images of HFpEF-hvCTS with AAV-GFP or AAV1.SERCA2A. (B) Representative force tracing of HFpEF-hvCTS with AAV-GFP (solid line) or AAV1.SERCA2A (dashed line). (C) Systolic force. HFpEF-hvCTS with AAV-GFP (solid circles); HFpEF-hvCTS with AAV1.SERCA2A (open circles). (D) Contractile time and relaxation time of HFpEF-GFP and HFpEF-SERCA hvCTS with post-tracking measurement. HFpEF-hvCTS with AAV-GFP (solid circles); HFpEF-hvCTS with AAV1.SERCA2A (open circles). (C)-(D) n=5−8. Student t-test.

FIG. 6. The effect of ET-1 on hvCTS. (A) Representative images of hvCTS treated with 0-100 nM ET-1. (B) Representative post-tracking normalized force tracing of hvCTS. 0 nM ET-1 (black line); 30 nM ET-1 (gray line); 100 nM ET-1 (dashed line). (C) Systolic force. 0 nM ET-1 (black circles); 30 nM ET-1 (gray circles); 100 nM ET-1 (open circles). (D) Contractile time and relaxation time of control and ET-1 hvCTS with post-tracking measurement. 0 nM ET-1 (black circles); 30 nM ET-1 (gray circles); 100 nM ET-1 (open circles). (E) Effect of %strain on hvCTS stiffness, developed force, max dF/dt and max-dF/dt as measured using the isometric system. (C)-(D) n=4. Student t-test. (E) n=2. Multiple t-test. Control (solid circles); ET-1 (open circles). *p<0.05 and **p<0.01.

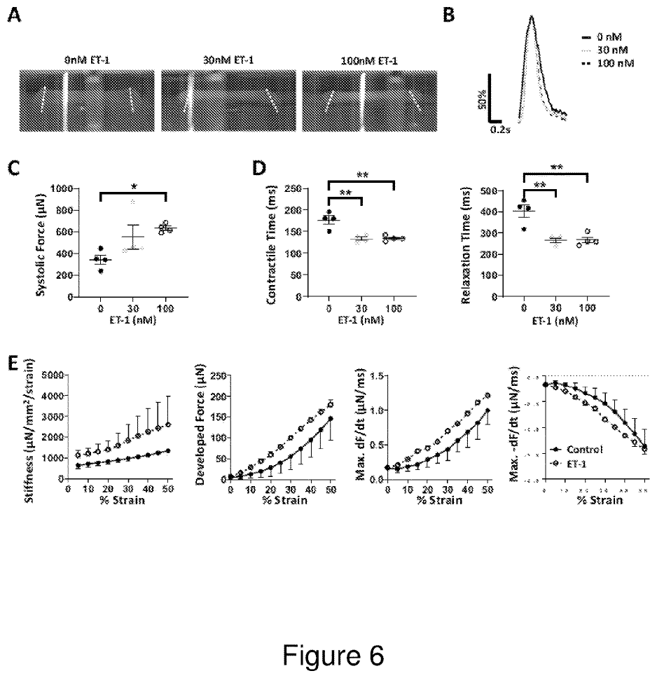

FIG. 7. The effect of TGF-β1 on hvCTS. (A) Representative images of hvCTS treated with 0-30 ng/ml TGF-β1. (B) Representative post-tracking normalized force tracing of hvCTS without TGF-β1 (Control; solid line) and 1 ng/ml TGF-β1 (dashed line). (C) Systolic force. 0 ng/ml TGF-β1 (solid circles); 1 ng/ml TGF-β1 (open circles); 3 ng/ml TGF-β1 (upright open triangles); 10 ng/ml TGF-β1 (inverted open triangles); 30 ng/ml TGF-β1 (open squares). (D) Contractile time and relaxation time of hvCTS with different TGF-β1 concentrations with post-tracking measurement. 0 ng/ml TGF-β1 (solid circles); 1 ng/ml TGF-β1 (open circles); 3 ng/ml TGF-β1 (upright open triangles); 10 ng/ml TGF-β1 (inverted open triangles); 30 ng/ml TGF-β1 (open squares). (E) Effect of %strain on hvCTS stiffness, developed force, max dF/dt and max-dF/dt of the Control and 1 ng/ml TGF-β1 groups as measured using the isometric system. Control (solid circles); TGF-β1 (open circles). (C)-(D) n=2-16. Student t-test. (E) n=12-16. Multiple t-test. *p<0.05, **p<0.01 and ***p<0.001.

### DETAILED DESCRIPTION

In mammalian hearts, HFpEF is associated with impaired cardiac relaxation [26, 27]. Cardiac myocytes isolated from HFpEF patients and experimental models are characterized by prolonged relaxation, diminished contraction velocity, a decrease in β-adrenergic response, and increased myocardial stiffness [26, 27]. A number of cellular and molecular mechanisms may contribute to the disease-related defects. It is believed that the abnormalities in cardiac relaxation were due to a defect in sarcoplasmic reticulum (SR) Ca²⁺-ATPase pump activity, which is mainly responsible for controlling the rate at which Ca²⁺ is taken up into the SR during relaxation [28-31]. The decrease in SR Ca²⁺uptake during relaxation, which results in prolonged contraction, has been associated with a decrease in the content and activity of the SR Ca²⁺-ATPase pump in experimental models of HFpEF.

Furthermore, SERCA2a protein levels were found to be significantly decreased in senescent human myocardium that is characterized by HFpEF. This decrease in SERCA2a levels was associated with impaired myocardial function at baseline and was further accentuated by higher heart rates and hypoxic conditions [32-34]. Studies of human left ventricular hypertrophy suggest a relationship between elevated calcium loads and diastolic tension which is amplified at faster heart rates. Calcium that is slow to dissociate from the troponin-actin-myosin complexes after contraction creates a “rigor-like” state that may contribute to increased diastolic stiffness. In addition, the slow re-uptake correlates directly with the relaxation phase of pressure. In experimental models of HFpEF, SERCA2a has been found to be decreased and intracellular calcium elevated. Thus, the consequence of slowed calcium re-uptake has implications not only for myocardial relaxation but also contributes to the development of diastolic stiffness, key features in the pathophysiology of HFpEF.

Abnormalities in lusitropy (myocardial relaxation), measured by impaired tissue relaxation, are evident both at rest and during exercise in the majority of HFpEF patients, although the sensitivity for diagnosis is lessened when solely measured at rest. During exercise, when preload and heart rate are both elevated, decreased distensibility is compounded by impaired lusitropy leading to rapid rises in left ventricular (L)V pressures in an incompletely relaxed ventricle. A number of cellular and molecular mechanisms may contribute to the disease-related defects. The abnormalities in cardiac relaxation have been attributed to a defect in sarcoplasmic reticulum (SR) Ca²⁺-ATPase pump activity, which is mainly responsible for controlling the rate at which Ca²⁺ is taken up into the SR during relaxation [28-31]. The decrease in SR Ca²⁺uptake during relaxation, which results in prolonged contraction and has been associated with a decrease in the content and activity of the SR Ca²⁺-ATPase pump in experimental models of HFpEF. Furthermore, SERCA2a protein levels were found to be significantly decreased in senescent human myocardium that was characterized by HFpEF. This decrease in SERCA2a levels was associated with impaired myocardial function at baseline and was further accentuated by higher heart rates and hypoxic conditions [32-34]. Another approach to restore intracellular calcium homeostasis is to enhance calcium uptake by SERCA. This can be achieved by modulating SERCA activity or increasing expression of SERCA pumps. We have shown that in HFpEF animal models which are characterized by abnormal lusitropy and a decrease in SERCA2a, delivery of SERCA2a by gene transfer resulted in restoration of relaxation parameters such as -dP/dt, the left ventricular time constant of isovolumic (i.e., isovolumetric) relaxation, tau, and the passive stiffness of the left ventricle to adult levels [31]. These results establish that viral delivery of SERCA2a in HFpEF hearts can improve relaxation parameters [31]. In other studies the Otsuka-Long-Evans Tokushima Fatty rat, which represents a model for spontaneous non-insulin-dependent type II diabetes mellitus (DM), is characterized by diastolic dysfunction, and is associated with decreased SERCA2a expression, was used as a model of HFpEF. In multiple studies using short-term expression of SERCA2a, oxygen consumption and relaxation parameters were restored to normal levels in these HFpEF models [28-30]. The experiments disclosed herein establish the HFpEF-hvCTS and HF-EF-hvCOC models of heart failure with preserved ejection fraction in human diseased hearts and the data further led to treatment methods for HFpEF involving increasing, or restoring, intracellular calcium homeostasis in cardiomyocytes by increasing or enhancing calcium uptake by SERCA to ameliorate or reverse HFpEF, for example in humans.

There currently are no human clinical trials targeting relaxation in HFpEF. To date only two clinical trials, both in chronic systolic heart failure (HFrEF), have tested the effects of improving SERCA2a function in HF. The HORIZONS-HF study investigated the effects of istaroxime, an intravenous agent that increases SERCA2a activity, in patients hospitalized with acute decompensated heart failure. Patients who received a 6-hour infusion of istaroxime had a 3-5 mmHg reduction in pulmonary capillary wedge pressure (PCWP) without changes in right atrial pressure or cardiac index [35]. The CUPID-1 study tested the effect of SERCA2a gene therapy in patients with HFrEF. Subjects randomized to the highest dose of a single intra-coronary infusion of AAV1-SERCA2a showed less deterioration in 6-minute walk time, peak VO₂ and NT-proBNP levels after 6 months [36-38]. In larger follow-up studies testing only this high dose (10¹³ viral genomes particles of AAV1.SERCA2a per patient), the safety of this approach was confirmed [39-40]. There was no effect of SERCA2a transduction on the primary outcome of HF hospitalization or decompensated HF, however. In patients with cardiac tissue available, the median AAV1-SERCA2a DNA level was measured at 43 copies/μg DNA (range 10-192 copies/μg DNA) which was below the doses of 2,000-50,000 copies/μg DNA that were effective in pre-clinical models. This very low transduction efficiency likely explained the neutral outcome [41]. Although results from the study were disappointing, the CUPID-2 trial's strategy was to increase SERCA2a activity as a means of improving contractile dysfunction that is characteristic of HFrEF. The effect of SERCA2a activity on cardiac relaxation was not assessed. Increasing SERCA2a activity or expression is expected to be an appealing therapeutic target in HFpEF. Indeed, the use of the human heart tissue and chambers disclosed herein enable us to titrate the optimized titers for transducing the human heart, which are much higher than those that work in animals. Shortening LV tau would likely allow for tolerance of higher heart rates by tempering rapid rises in ventricular chamber pressures, allowing more complete filling of the ventricles with blood, and improving exercise capacity and peak VO₂. Therefore, transduction with higher doses of AAV1.SERCA2A is also expected to lead to more effective and robust dose-response. Further, increasing or restoring SERCA protein expression is expected to lead, over time, to reversal of other maladaptive pathways as increased SERCA2a activity restores myocardial calcium homeostasis. Taking the above together with the observation disclosed herein that higher viral titers were beneficial in transducing hCMs, it is expected that higher doses of AAV1.SERCA2a than were used in CUPID2 will provide an effective treatment for patients with HFpEF with precise characterization of myocardial relaxation and filling pressures during exercise before and after treatment.

In sum, the disclosed HFpEF-hvCTS and HFpEF-hvCOC are of human origin in the disease setting (HFpEF), and amenable to sophisticated phenotypic measurements, as revealed in the following Examples. Thus, the HFpEF-hvCTS and hvHFpEF-hvCOC heart models were able to recapitulate characteristic phenotypes seen in patients. Using the SERCA2a transgene as an example, we also demonstrated how the disclosed human HFpEF models are used to identify novel druggable targets followed by therapeutic screening for cardiac improvements. The versatile HFpEF models can be further custom-tailored, as needed, for discovering additional novel targets and screening therapeutics.

Collectively, the preclinical human HFpEF models disclosed herein are useful in investigating disease mechanisms, and for facilitating the discovery of novel druggable targets and screening of therapeutics to identify the most promising agent(s) by enabling accurate predictions of the clinical effectiveness and safety of new medicines before human trials.

### EXAMPLES

### Example 1

### Materials and Methods

Induction of HFpEF in Human Ventricular Cardiac Tissue Strips (hvCTS)

Three-dimensional (3D) multicellular human ventricular cardiac tissue strips (hvCTSs) comprising myocardial tissues were engineered in a manner consistent with previous descriptions ([6, 10]). Briefly, cardio-clusters from day 15 of hPSC cardiac differentiation were dissociated into single cells and allowed to recover in the incubator for 3 days before hvCTS construction. Each hvCTS consisted of 1.3×10⁶ cardiac cells differentiated from hPSCs and 1.3×10⁵ human foreskin fibroblasts in a 100-μl ice-cold solution of 2 mg/ml collagen |(0.80-0.95 mg/ml Matrigel, 0.6X PBS, 20 mM NaOH, 0.8X Minimum Essential Medium (Sigma-Aldrich), 1.6 mM HEPES, and 0.1X hvCTS maintenance medium (see composition below). A volume of 100 ul of the final cell collagen mixture was then added to each polydimethylsiloxane (PDMS) bioreactor, consisting of a force-sensing cantilever post at each end of a rectangular well, and returned to the incubator to form the hvCTS attached between the two end posts. The hvCTSs were maintained in DMEM medium supplemented with 10% newborn calf serum (Gibco), with daily half-medium changes for 5 days, then switched to RPMI+B27 with TGFβ1 (1 ng/ml) and ET-1 (100 nM) for a further 5.5 days to induce the HFpEF phenotype, yielding hvCTS with the HFpEF phenotype ready for testing.

Measurement of Stress and Strain in hvCTS

Force generated by the hvCTS was measured at 37 °C in phenol red-free DMEM medium with HEPES buffer using an isometric muscle bath system. Developed force and other contractile parameters were acquired under spontaneous (unpaced) and electrically paced conditions (field stimulation at 1.0, 1.5, 2.0, 2.5 and 3.0 Hz) at 0-50% of natural length of the tissue (L0) at 5% intervals, using the isometric muscle bath system. Stiffness was calculated as stress/strain.

HFpEF Induction in Human Cardiac Organoid Chambers (hvCOC)

3D multi-cellular human ventricular cardiac organoid chamber (hvCOC) myocardial tissues were engineered as previously described ([8], [42]) with an ultra-compliant indwelling elastomer balloon. For ease of exposition, hvCOC is used herein to refer to human ventricular cardiac organoid chambers and to the human ventricular cardiac organoids contained in such chambers, as would be apparent from context. Briefly, each hvCOC consisted of 1.0×10⁷ cardiac cells differentiated from hPSCs and 1.0×10⁶ human foreskin fibroblasts in a 1650-pL ice-cold solution of 2 mg/ml collagen 1,0.80-0.95 mg/ml Matrigel, 0.6X PBS, 20 mM NaOH, 0.8× Minimum Essential Medium, 1.6 mM HEPES, and 0.1× hvCTS maintenance medium (see composition below). The cell collagen mix was added to the space between the agarose mold and the balloon, ensuring that the porous polyethylene ring was placed just below an O-ring (5.8 mm) 3 mm above the base of the balloon to enhance tissue attachment, and was submerged in the cell suspension. The bioreactor was incubated for 1 hour for gel solidification before being topped off with NCS medium (8 mL) to reach the top of the bioreactor. Medium was changed every 24 hours while the hvCOC was compacting. The medium change was done every other day after the hvCOC was removed from the agarose gel for 5 days, switched to RPMI+B27 with TGFβ1 (1 ng/ml) for 4 days and to RPMI+B 27 with TGFβ1 (1 ng/ml)+ET-1 (100nM) for a further 1.5 days to induce the HFpEF phenotype.

Measurement of Stiffness and Function in hvCOC

A high-sensitivity pressure catheter was advanced into the lumen of the hvCOC chamber for pressure measurements. A digital camera was mounted outside of the bioreactor and permitted direct tissue monitoring for determination of chamber area.

Chamber pressure and digital video were acquired simultaneously under spontaneous (unpaced) and electrically paced conditions (field stimulation at 1.0, 1.5, 2.0, 2.5 and 3.0 Hz) at 0, 25, 50, 100 L loading. Stiffness was calculated by plotting the slope of change in diastolic pressure as a function of the change in diastolic area of the chamber.

Bulk RNA Sequencing of HFpEF hvCOC and hvCTS

Differentially expressed genes (DEGs) from RNAseq analyses of HFpEF-hvCTS and HFpEF-hvCOC models (TGFB1+ET1 versus control) were compared to human heart failure transcriptomic data [11] for gene-level analyses and comparative functional enrichment analyses.

AAV1.SERCA2A Infection in hvCTS

SERCA2a mRNA was delivered into hvCTS by transduction with AAV1.SERCA2A (comprising an expressible coding region for SERCA2a) into dissociated human pluripotent stem cell-derived cardiomyocytes (hPSC-CMs) at day 15 post-differentiation at 1×10² viral genomes (vg) to 1×10⁵ vg per cardiomyocyte. The hPSC-CMs were then fabricated into hvCTS, HFpEF induced with HFpEF induction medium containing TGFB1+ET1, as described above, and maintained in such medium until ready for testing at day 11 post-fabrication. In some experiments, the SERCA2a mRNA was delivered into the hvCTS without HFpEF induction by directly transducing the hvCTS with the AAV1.SERCA2A and maintained for 8 days until ready for testing.

### Example 2

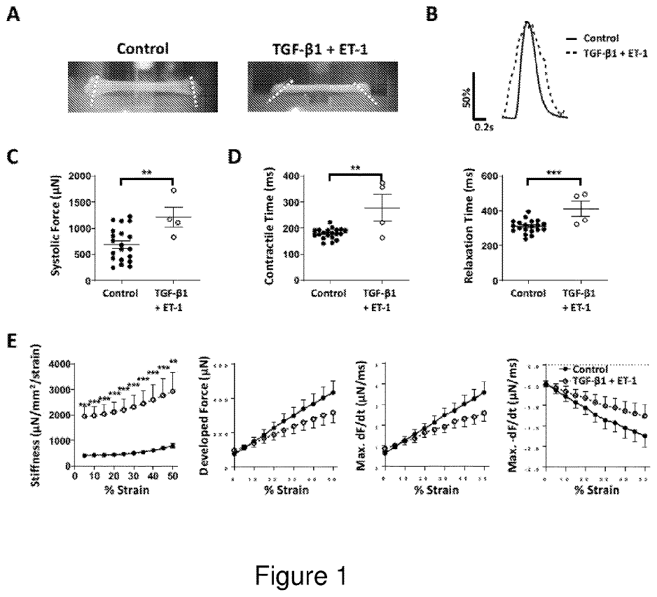

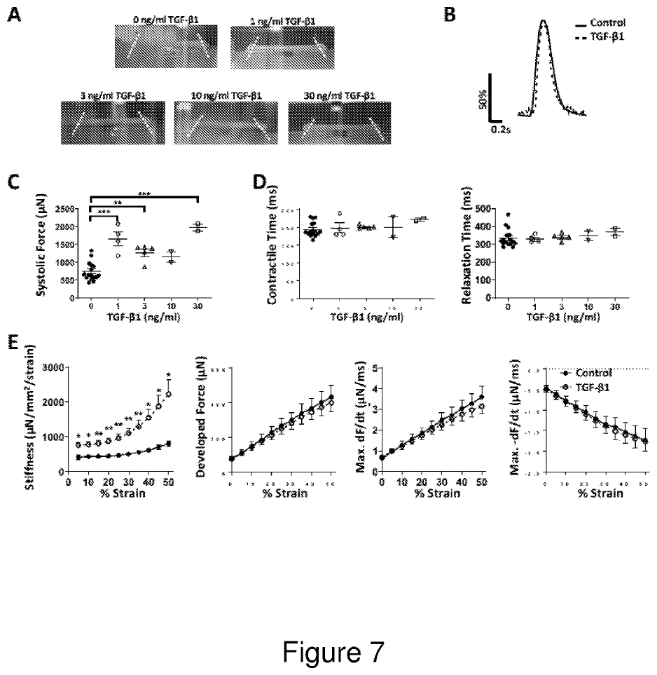

hvCTS Exhibiting a HFpEF Phenotype po Given the biological role of ET-1 and TGF-β1 in HFpEF, we tested the combination of a hypertrophic stimulator and a pro-fibrotic signal to reproduce the HFpEF phenotype by examining the functional consequences of their combined effect on the contractility of hvCTS, a concern relevant to the creation of a human HFpEF heart model. FIG. 1A shows the representative images of time-matched hvCTS under control conditions and after combined ET-1/TGF-β1 treatment (5.5 days). Of note, the posts for the treated group were bent, indicating a higher passive strain of the corresponding mounted hvCTS. Consistently, the ET-1/TGF-β1-treated group displayed slower contractile and relaxation kinetics compared to control as assessed by post-tracking measurements (FIG. 1B-D; n=4−20, **p<0.01 and ***p<0.001). For further mechanistic insights, recordings of hvCTS using an isometric system for length-control were obtained. FIG. 1E shows the relationship of stiffness, developed force, max dF/dt and max-dF/dt as a function of % strain for control and ET-1/TGF-β1-treated hvCTS (n=7−12). Collectively, these data showed that ET-1/TGF-β1-treated, but not control or singly-treated (FIGS. 7 and 8), hvCTS uniquely displayed significantly increased stiffness, along with slowed relaxation and contraction kinetics, all of which were characteristics observed in HFpEF patients.

### Example 3

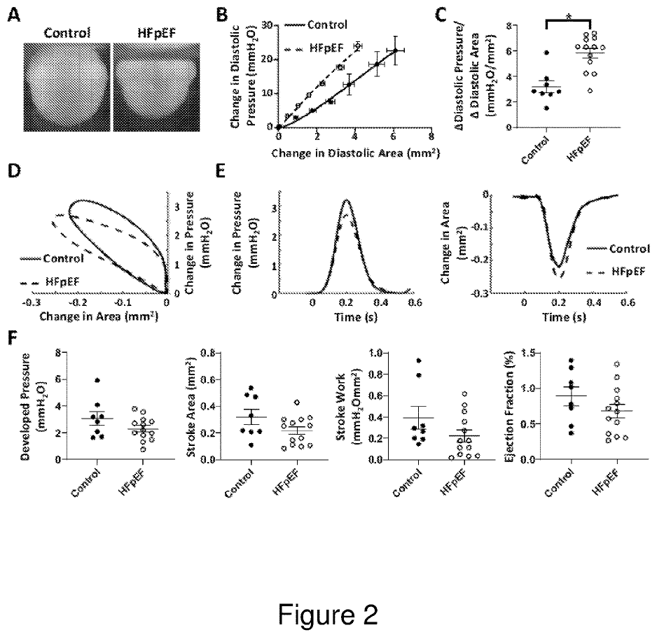

ET-1/TGF-β1 administration was then assessed for the ability to confer HFpEF phenotypic characteristics on hvCOC (i.e., the hv cardiac organoids contained in hvCOCs). FIG. 2A shows the representative images of time-matched hvCOC under control conditions and after combined ET-1/TGF-β1 treatment. FIG. 2B shows that chamber stiffness, as deduced from the plot of the changes in diastolic pressure against the changes in diastolic area after hydrostatic loading (100 μL), was significantly higher in ET-1/TGF-β1-treated hvCOC, along with a more compact appearance, compared to time-matched control hvCOCs. Representative pressure-volume loops of control (black) and ET-1/TGF-β1-treated (red) hvCOCs are given in FIG. 2D. Despite the increased stiffness, no changes in the developed pressure, stroke volume, stroke work or ejection fraction were observed between control (black) and ET-1/TGF-β1-treated (red) hvCOCs, indicating that the contractile function was not significantly altered. Therefore, ET-1/TGF-β1-treated hvCOC displayed phenotypes consistent with HFpEF characteristics (FIG. 2 E-F; n=8−13, *p<0.05).

### Example 4

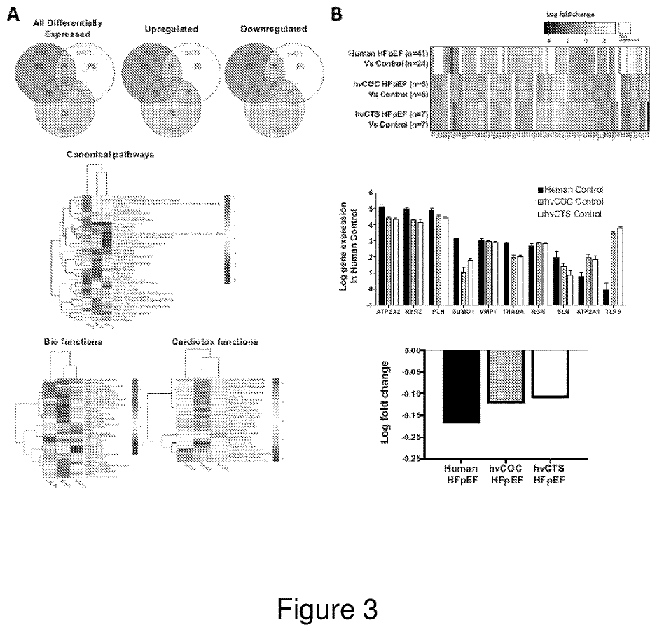

To obtain molecular insights into HFpEF tissue, we performed transcriptomic and bioinformatic analyses to systematically compare normal and HFpEF patients, as well as control and HFpEF-induced hvCTS and hvCOC (FIG. 3). The data were organized into a Venn diagram that showed that there was a 29% overlap of differentially expressed genes (DEG) between HFpEF patients and the HFpEF-hvCTS model (FIG. 3A). This overlap was 33% between HFpEF patients and the HFpEF-hvCOC model. Comparative functional analyses using Ingenuity Pathway Analysis further revealed that the HFpEF-hvCOC model was more similar to human HFpEF patients than the HFpEF-hvCTS model based on hierarchical clustering of enriched canonical pathways, biofunctions and cardiotox functions (FIG. 3B). Interestingly, the calcium signaling pathway was similarly enriched in both human HFpEF patients and engineered HFpEF-hvCTS and HFpEF-hvCOC models relative to their corresponding controls. Of note, the ATP2A2 gene (encoding for SERCA2a) was the most highly expressed gene in the GO term “Regulation of ATPase-coupled calcium transmembrane transport activity” in normal patients as well as control engineered hvCTS and hvCOC. Interestingly, ATP2A2 (SERCA2a) was also most significantly down-regulated in HFpEF patients and engineered HFpEF-hvCTS and HFpEF-hvCOC models compared to their respective controls. These transcriptomic and bioinformatic results were consistent with the phenotypes observed in HFpEF patients, indicating that SERCA2a is a target candidate for rescuing or reversing the disease traits.

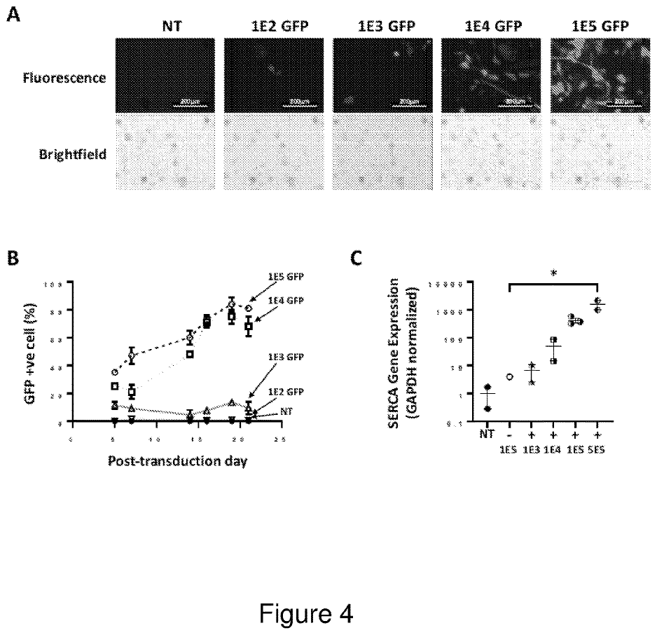

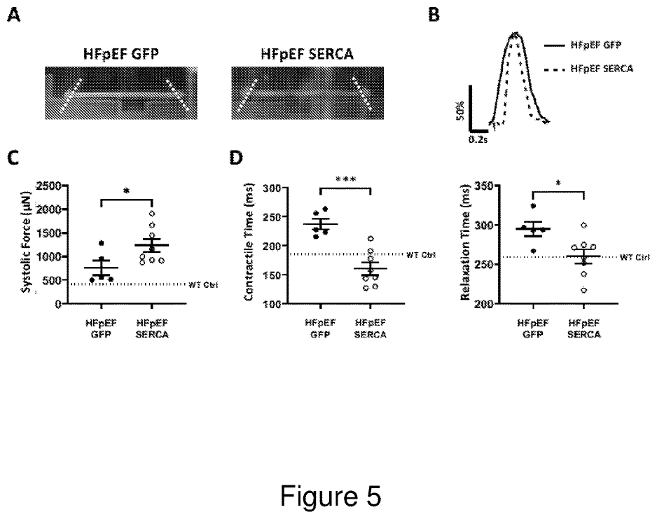

To test this hypothesis, we started by investigating the titer-and time-dependences of recombinant adeno-associated vector type 1 (AAV1) transduction of hCMs. FIG. 4A shows that the percentage of Green Fluorescent Protein-positive (GFP-positive) hvCMs after AAV-GFP transduction gradually increased over time until a plateau was reached (FIG. 4 A and B). The time it took to reach the plateau and the plateau level were dependent on the viral titer (viral genome or vg per cell). Similarly, the effect on SERCA2a gene expression after 14 days of AAV 1-SERCA2a transduction was both time-and titer-dependent (n=1−3; One-way ANOVA, *p<0.05). Based on these data, we selected a titer of 1×10⁵ viral genomes per cell and day 14 for studying the effects of AAV1-SERCA2a-mediated overpression in HFpEF-hvCTS. FIG. 5A shows representative images of time-matched hvCOC under HFpEF conditions and after combined AAV1.SERCA2a treatment. FIGS. 5C-D shows that AAV1-SERCA2a transduction rescued the disease phenotype in HFpEF-hvCTS by restoring the slowed contractile kinetics. Isometric measurements further revealed the effect of % strain on the stiffness, developed force, max dF/dt and max-dF/dt of AAV1.SERCA2A-transduced HFpEF-hvCTS (n=5−8, p<0.05).

### REFERENCES

1. Shah, A. M., et al., Prognostic Importance of Impaired Systolic Function in Heart Failure With Preserved Ejection Fraction and the Impact of Spironolactone.
Circulation, 2015. 132(5): p. 402-14.
2. Shah, S. J., et al., Phenomapping for novel classification of heart failure with preserved ejection fraction. Circulation, 2015. 131(3): p. 269-79.
3. Shah, S. J., et al., Phenotype-Specific Treatment of Heart Failure With Preserved Ejection Fraction: A Multiorgan Roadmap. Circulation, 2016. 134(1): p. 73-90.
4. Virani, S. S., et al., Heart Disease and Stroke Statistics-2020 Update: A Report From the American Heart Association. Circulation, 2020. 141(9): p. e 139-e596.
5. Weng, Z., et al., A simple, cost-effective but highly efficient system for deriving ventricular cardiomyocytes from human pluripotent stem cells. Stem Cells Dev, 2014. 23(14): p. 1704-16.
6. Cashman, T. J., et al., Construction of Defined Human Engineered Cardiac Tissues to Study Mechanisms of Cardiac Cell Therapy. J Vis Exp, 2016(109): p. e 53447.
7. Keung, W., et al., Human Cardiac Ventricular-Like Organoid Chambers and Tissue Strips From Pluripotent Stem Cells as a Two-Tiered Assay for Inotropic Responses. Clin Pharmacol Ther, 2019. 106(2): p. 402-414.
8. Li, R. A., et al., Bioengineering an electro-mechanically functional miniature ventricular heart chamber from human pluripotent stem cells. Biomaterials, 2018. 163: p. 116-127.
9. Wong, A. O., et al., Adverse effects of hydroxychloroquine and azithromycin on contractility and arrhythmogenicity revealed by human engineered cardiac tissues. J Mol Cell Cardiol, 2021. 153: p. 106-110.
10. Turnbull, I. C., et al., Advancing functional engineered cardiac tissues toward a preclinical model of human myocardium. FASEB J, 2014. 28(2): p. 644-54.
11. Hahn, V. S., et al., Myocardial Gene Expression Signatures in Human Heart Failure With Preserved Ejection Fraction. Circulation, 2021. 143(2): p. 120-134.
12. Sharma, K. and D. A. Kass, Heart failure with preserved ejection fraction: mechanisms, clinical features, and therapies. Circ Res, 2014. 115(1): p. 79-96.
13. Bornstein, P. and E. H. Sage, Matricellular proteins: extracellular modulators of cell function. Curr Opin Cell Biol, 2002. 14(5): p. 608-16.
14. Chen, M. M., et al., CTGF expression is induced by TGF-beta in cardiac fibroblasts and cardiac myocytes: a potential role in heart fibrosis. J Mol Cell Cardiol, 2000. 32(10): p. 1805-19.
15. Frey, N. and E. N. Olson, Cardiac hypertrophy: the good, the bad, and the ugly. Annu Rev Physiol, 2003. 65: p. 45-79.
16. Gao, X. M., et al., Regression of pressure overload-induced left ventricular hypertrophy in mice. Am J Physiol Heart Circ Physiol, 2005. 288(6): p. H 2702-7.
17. Hunter, J. J. and K. R. Chien, Signaling pathways for cardiac hypertrophy and failure. N Engl J Med, 1999. 341(17): p. 1276-83.
18. Molkentin, J. D. and G. W. Dorn, 2nd, Cytoplasmic signaling pathways that regulate cardiac hypertrophy. Annu Rev Physiol, 2001. 63: p. 391-426.
19. Sabbah, M., et al., CCN5, a novel transcriptional repressor of the transforming growth factor beta signaling pathway. Mol Cell Biol, 2011. 31(7): p. 1459-69.
20. Yang, D. K., et al., Gene profiling during regression of pressure overload-induced cardiac hypertrophy. Physiol Genomics, 2007. 30(1): p. 1-7.
21. Yoon, P. O., et al., The opposing effects of CCN 2 and CCN 5 on the development of cardiac hypertrophy and fibrosis. J Mol Cell Cardiol, 2010. 49(2): p. 294-303.
22. Chowdhury, M. A., et al., Endothelin 1 Is Associated with Heart Failure Hospitalization and Long-Term Mortality in Patients with Heart Failure with Preserved Ejection Fraction and Pulmonary Hypertension. Cardiology, 2019. 143(3-4): p. 124-133.
23. Widyantoro, B., et al., Endothelial cell-derived endothelin-1 promotes cardiac fibrosis in diabetic hearts through stimulation of endothelial-to-mesenchymal transition. Circulation, 2010. 121(22): p. 2407-18.
24. Dobaczewski, M., et al., Smad 3 signaling critically regulates fibroblast phenotype and function in healing myocardial infarction. Circ Res, 2010. 107(3): p. 418-28.
25. Oberhaus, S. M., TUNEL and immunofluorescence double-labeling assay for apoptotic cells with specific antigen(s). Methods Mol Biol, 2003. 218: p. 85-96.
26. Zile, M. R., et al., Myocardial stiffness in patients with heart failure and a preserved ejection fraction: contributions of collagen and titin. Circulation, 2015. 131(14): p. 1247-59.
27. Zile, M. R., et al., Prevalence and significance of alterations in cardiac structure and function in patients with heart failure and a preserved ejection fraction. Circulation, 2011. 124(23): p. 2491-501.
28. Sakata, S., et al., Restoration of mechanical and energetic function in failing aortic-banded rat hearts by gene transfer of calcium cycling proteins. J Mol Cell Cardiol, 2007. 42(4): p. 852-61.
29. Sakata, S., et al., Transcoronary gene transfer of SERCA 2a increases coronary blood flow and decreases cardiomyocyte size in a type 2 diabetic rat model. Am J Physiol Heart Circ Physiol, 2007. 292(2): p. H 1204-7.
30. Sakata, S., et al., Mechanical and metabolic rescue in a type II diabetes model of cardiomyopathy by targeted gene transfer. Mol Ther, 2006. 13(5): p. 987-96.
31. Schmidt, U., et al., Restoration of diastolic function in senescent rat hearts through adenoviral gene transfer of sarcoplasmic reticulum Ca(2+)-ATPase. Circulation, 2000. 101(7): p. 790-6.
32. Pabel, S., et al., Empagliflozin directly improves diastolic function in human heart failure. Eur J Heart Fail, 2018. 20(12): p. 1690-1700.
33. Ahmad, S., et al., The functional consequences of sodium channel NaV 1.8 in human left ventricular hypertrophy. ESC Heart Fail, 2019. 6(1): p. 154-163.
34. Kolijn, D., et al., Empagliflozin improves endothelial and cardiomyocyte function in human heart failure with preserved ejection fraction via reduced pro-inflammatory-oxidative pathways and protein kinase Galpha oxidation. Cardiovasc Res, 2021. 117(2): p. 495-507.
35. Shah, S. J., et al., Effects of istaroxime on diastolic stiffness in acute heart failure syndromes: results from the Hemodynamic, Echocardiographic, and Neurohormonal Effects of Istaroxime, a Novel Intravenous Inotropic and Lusitropic Agent: a Randomized Controlled Trial in Patients Hospitalized with Heart Failure (HORIZON-HF) trial. Am Heart J, 2009. 157(6): p. 1035-41.
36. Jaski, B. E., et al., Calcium upregulation by percutaneous administration of gene therapy in cardiac disease (CUPID Trial), a first-in-human phase 1/2 clinical trial. J Card Fail, 2009. 15(3): p. 171-81.
37. Jessup, M., et al., Calcium Upregulation by Percutaneous Administration of Gene Therapy in Cardiac Disease (CUPID): a phase 2 trial of intracoronary gene therapy of sarcoplasmic reticulum Ca2+-ATPase in patients with advanced heart failure. Circulation, 2011. 124(3): p. 304-13.
38. Zsebo, K., et al., Long-term effects of AAV 1/SERCA2a gene transfer in patients with severe heart failure: analysis of recurrent cardiovascular events and mortality. Circ Res, 2014. 114(1): p. 101-8.
39. Hulot, J. S., et al., Effect of intracoronary administration of AAV 1/SERCA2a on ventricular remodelling in patients with advanced systolic heart failure: results from the AGENT-HF randomized phase 2 trial. Eur J Heart Fail, 2017. 19(11): p. 1534-1541.
40. Greenberg, B., et al., Calcium upregulation by percutaneous administration of gene therapy in patients with cardiac disease (CUPID 2): a randomised, multinational, double-blind, placebo-controlled, phase 2 b trial. Lancet, 2016. 387(10024): p. 1178-86.
41. Hulot, J. S., K. Ishikawa, and R. J. Hajjar, Gene therapy for the treatment of heart failure: promise postponed. Eur Heart J, 2016. 37(21): p. 1651-8.
42. Roberts et al, Ultra-Compliant Indwelling Elastomer Balloons Improve Stability and Performance of Bioengineered Human Mini-Hearts, Adv Eng Mater, 2022. Wiley online library (https://doi.org/10.1002/adem.202101481).

All patents and other publications identified are expressly incorporated herein by reference in their entireties for the purpose of describing and disclosing, for example, the methodologies described in such publications that might be used in connection with information described herein.

What is claimed is:

1. A method for identifying a therapeutic for treatment of heart failure with preserved ejection fraction, the method comprising:
(a) contacting cardiac tissue with an effective amount of Transforming Growth Factor-β1 and an effective amount of Endothelin-1 (ET-1) to induce heart failure with preserved ejection fraction in the cardiac tissue;
(b) exposing the cardiac tissue exhibiting heart failure with preserved ejection fraction to a candidate therapeutic, and
(c) identifying the candidate therapeutic as a therapeutic for heart failure with preserved ejection fraction if the level of (i) passive strain of the cardiac tissue, (ii) cardiac tissue stiffness, (iii) cardiac myocyte hypertrophy, or (iv) cardiac fibrosis, is reduced, or the (v) contractile kinetics, or (vi) relaxation kinetics are increased, or any combination thereof, in the presence of the candidate therapeutic compared to the absence of the candidate therapeutic.

2. The method of claim 1 wherein the cardiac tissue is formed into a cardiac tissue strip.

3. The method of claim 2 wherein the cardiac tissue strip is formed between two posts and the reduced level of passive strain is detected by a reduction in the bending of the cardiac tissue strip.

4. The method of claim 1 wherein the candidate therapeutic reduces the passive strain of the cardiac tissue.

5. The method of claim 1 wherein the candidate therapeutic reduces the tissue stiffness of the cardiac tissue.

6. The method of claim 1 wherein the candidate therapeutic reduces cardiac myocyte hypertrophy in the cardiac tissue.

7. The method of claim 1 wherein the candidate therapeutic reduces cardiac fibrosis in the cardiac tissue.

8. The method of claim 1 wherein the candidate therapeutic increases the contractile kinetics of the cardiac tissue.

9. The method of claim 1 wherein the candidate therapeutic increases the relaxation kinetics of the cardiac tissue.

10. The method of claim 1 wherein the candidate therapeutic is a small molecule, a nucleic acid, or a cell.

11. A method for identifying a therapeutic for treatment of heart failure with preserved ejection fraction, the method comprising:
(a) contacting a cardiac organoid with an effective amount of Transforming Growth Factor-31 and an effective amount of Endothelin-1 (ET-1) to induce heart failure with preserved ejection fraction in the cardiac tissue;
(b) exposing the cardiac organoid exhibiting heart failure with preserved ejection fraction to a candidate therapeutic, and
(c) identifying the candidate therapeutic as a therapeutic for heart failure with preserved ejection fraction if the level of (i) passive strain of the cardiac organoid, (ii) stiffness of cardiac tissue in the cardiac organoid, (iii) cardiac myocyte hypertrophy in the cardiac organoid, or (iv) fibrosis in the cardiac organoid, is reduced, or the (v) contractile kinetics of the cardiac organoid, or (vi) relaxation kinetics of the cardiac organoid are increased, or any combination thereof, in the presence of the candidate therapeutic compared to the absence of the candidate therapeutic.

12. The method of claim 11 wherein the candidate therapeutic reduces the passive strain of the cardiac organoid.

13. The method of claim 11 wherein the candidate therapeutic reduces the stiffness of the cardiac organoid.

14. The method of claim 11 wherein the candidate therapeutic reduces cardiac myocyte hypertrophy in the cardiac organoid.

15. The method of claim 11 wherein the candidate therapeutic reduces cardiac fibrosis in the cardiac organoid.

16. The method of claim 11 wherein the candidate therapeutic increases the contractile kinetics of the cardiac organoid.

17. The method of claim 11 wherein the candidate therapeutic increases the relaxation kinetics of the cardiac organoid.

18. The method of claim 11 wherein the candidate therapeutic is a small molecule, a nucleic acid, or a cell.

19. A method of assessing the toxicity of a compound to a cardiomyocyte, comprising:
(a) contacting cardiac tissue with an effective amount of Transforming Growth Factor-31 and an effective amount of Endothelin-1 (ET-1) to induce heart failure with preserved ejection fraction in the cardiac tissue;
(b) exposing the cardiac tissue exhibiting heart failure with preserved ejection fraction to a compound, and
(c) determining or confirming the toxicity of the compound to cardiac tissue if the level of (i) passive strain of the cardiac tissue, (ii) cardiac tissue stiffness, (iii) cardiac myocyte hypertrophy, or (iv) cardiac fibrosis, is reduced, or the (v) contractile kinetics, or (vi) relaxation kinetics are increased, or any combination thereof, in the presence of the compound compared to the absence of the compound.

20. The method of claim 19 wherein the toxicity of the compound to the cardiac tissue is determined.

21. The method of claim 19 wherein the cardiac tissue is a cardiac tissue strip.

22. The method of claim 19 wherein the cardiac tissue is a cardiac organoid.

23. A method of determining the efficacy of a compound in reducing at least one symptom of heart failure with preserved ejection fraction, the method comprising:
(a) contacting cardiac tissue with an effective amount of Transforming Growth Factor-β1 and an effective amount of Endothelin-1 (ET-1) to induce heart failure with preserved ejection fraction in the cardiac tissue;
(b) exposing the cardiac tissue exhibiting heart failure with preserved ejection fraction to a compound, and
(c) determining or confirming the efficacy of the compound in reducing at least one symptom of heart failure with preserved ejection fraction in the cardiac tissue if the level of (i) passive strain of the cardiac tissue, (ii) cardiac tissue stiffness, (iii) cardiac myocyte hypertrophy, or (iv) cardiac fibrosis, is reduced, or the (v) contractile kinetics, or (vi) relaxation kinetics are increased, or any combination thereof, in the presence of the compound compared to the absence of the compound.

24. The method of claim 23 wherein the efficacy of the compound in reducing at least one symptom of heart failure with preserved ejection fraction is determined.

25. The method of claim 23 wherein the cardiac tissue is a cardiac tissue strip.

26. The method of claim 23 wherein the cardiac tissue is a cardiac organoid.
